(19) United States
(12) Patent Application Publication
(10) Pub. No.: US 2008/0161923 A1
(43) Pub. Date: Jul. 3, 2008

(54) Intervertebral Motion Disc Having A Resorbable Keel

(76) Inventor: Matthew Parsons, Dartmouth, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/536,381

(22) Filed: Sep. 28, 2006

(51) Int. Cl. A61F 2/44

(52) U.S. Cl. 623/17.14

(57) ABSTRACT

An intervertebral motion disc having an at least partially resorbable keel.

### BACKGROUND OF THE INVENTION

The leading cause of lower back pain arises from rupture or degeneration of lumbar intervertebral discs. Pain in the lower extremities is caused by the compression of spinal nerve roots by a bulging disc, while lower back pain is caused by collapse of the disc and by the adverse effects of articulation and weight-bearing through a damaged, unstable vertebral joint. One proposed method of managing these problems is to remove the problematic disc and replace it with a prosthetic disc that allows for natural motion between the adjacent vertebrae (“a motion disc”).

Some such intervertebral motion discs use large fins or “keels”, embedded into the vertebral bodies for fixation. Keels have the advantage of excellent immediate resistance to implant migration from the disc space (prior to bony apposition on an implant coating), but the disadvantage of potentially compromising vertebral body integrity. In some instances, the implantation of motion discs having keels at adjacent levels have been reported to split vertebral bodies in half. Another potential disadvantage of keels is that they limit or eliminate the ability to revise a disc.

U.S. Pat. No. 7,060,073 (“Frey”) discloses an intervertebral cage that may be made from resorbable or non-resorbable materials.

U.S. Pat. No. 6,719,795 (“Cornwall”) discloses resorbable fixation implants having ‘rolled resorbable membranes’ for posterior fusion in the lateral gutters US Published Patent Application No. 2005/0240269 (“Lambrecht”) discloses an annular repair device having stabilizing projections. This device may be resorbable. Non-specific ‘support members’ and ‘anchors’ are described that may attach to bone or soft tissue to stabilize the annular repair device. These members may be resorbable, but are described as non-integral barbs or screws connected to the implant by wire or suture.

US Published Patent Application No. 2005/0187631 (“Van Hoeck”) discloses an artificial disc having a resorbable motion limiter and resorbable anchors.

### SUMMARY OF THE INVENTION

The present invention provides a completely or partially resorbable keel on an intervertebral implant. The resorbable keel provides the advantages of excellent fixation upon initial implantation without the long-term compromise to bony integrity and revisability.

Therefore, in accordance with the present invention, there is provided an intervertebral motion disc comprising an outer surface adapted to mate with a first vertebral body, wherein the outer surface has an at least partially resorbable keel extending therefrom.

### DESCRIPTION OF THE FIGURES

FIGS. 1a and 1b disclose front and side views of a motion disc of the present invention having a fully resorbable keel.

FIGS. 2a and 2b disclose front and side views of a motion disc of the present invention having a partially resorbable keel.

FIG. 2c discloses an endplate component of a motion disc of the present invention wherein the non-resorbable portion of the keel is somewhat smaller than the resorbable portion.

FIGS. 3a and 3b disclose a motion disc having a partially resorbable keel implanted within an intervertebral disc space at different time points.

FIG. 4 discloses a perspective view of a motion disc of the present invention having a keel having a porous, non-resorbable inner portion.

### DETAILED DESCRIPTION OF THE INVENTION

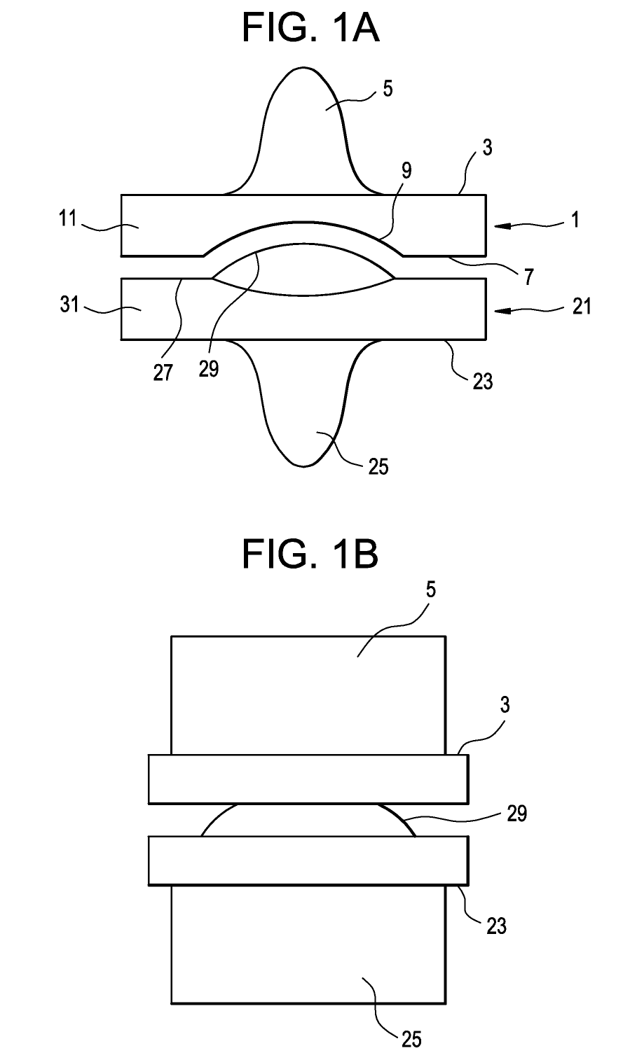

Now referring to FIGS. 1a and 1b, there is provided an articulating motion disc of two piece design that comprises:

a) a first prosthetic vertebral endplate 1 comprising:
i) an outer surface 3 adapted to mate with a first vertebral body, the outer surface having a fully resorbable keel 5 extending therefrom,
ii) an inner surface 7 having a first articulation surface 9, and
iii) a body portion 11 connecting the inner and outer surfaces, and

b) a second prosthetic vertebral endplate 21 comprising:
i) an outer surface 23 adapted to mate with a second vertebral body, the outer surface having a fully resorbable keel 25 extending therefrom,
ii) an inner surface 27 comprising a second articulation surface 29, and
iii) a body portion 31 connecting the inner and outer surfaces,
   wherein the first and second articulation surfaces are oriented to produce an articulation interface.

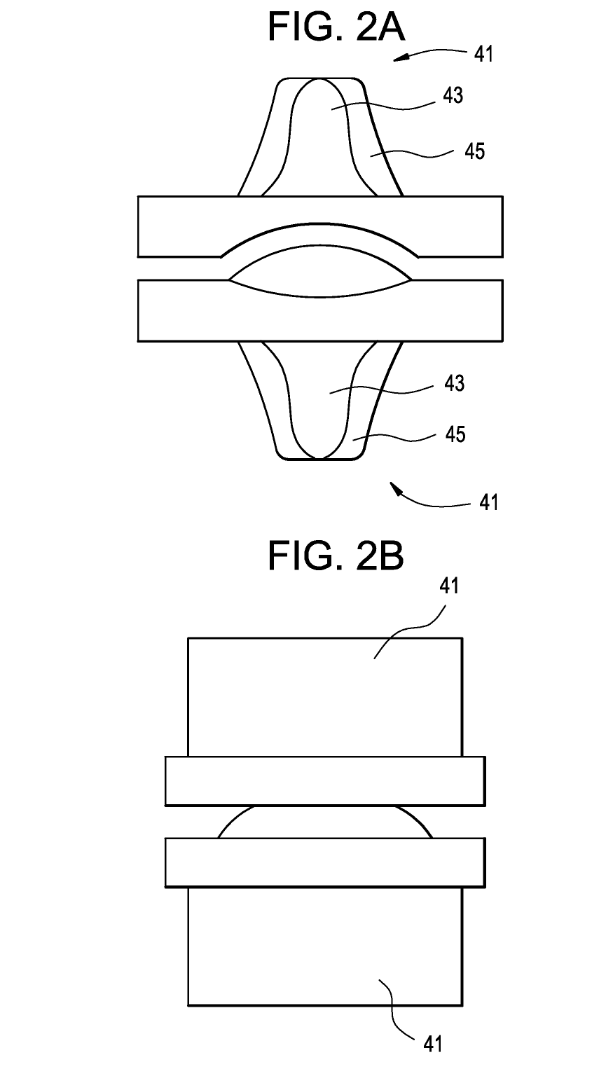

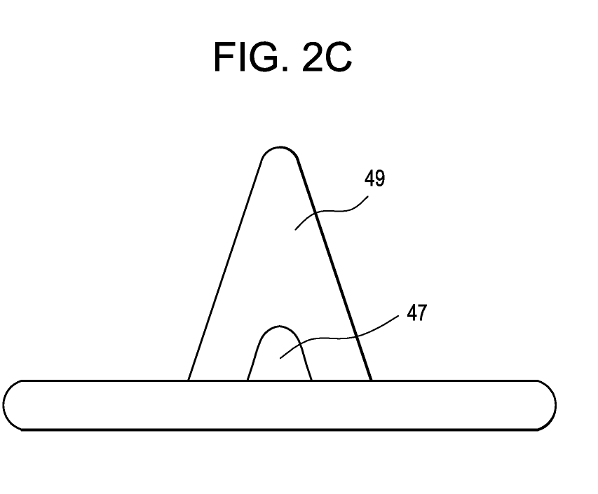

Now referring to FIG. 2a and 2b, there is provided a motion disc substantially similar to that of FIGS. 1a and 1b, except that the keel 41 is a partially resorbable keel, and comprises a non-resorbable inner portion 43 and a resorbable outer portion 45.

Now referring to FIG. 2c, there is provided an endplate of a motion disc, substantially similar to those of FIGS. 2a and 2b, except that the non-resorbable inner portion 47 of the keel is somewhat smaller than the resorbable outer portion 49. The large resorbable portion provides the short-term securement of a large keel until it resorbs and bone has attached to the surface of the endplate over a period of weeks. After that time the smaller non-resorbable portion would still provide some securement, but without compromising the integrity of the vertebral body as much as a larger keel. In preferred embodiments, the height of the non-resorbable inner portion of the keel is less than 50% of the height of the resorbable outer portion, more preferably less than 25%, more preferably less than 10%.

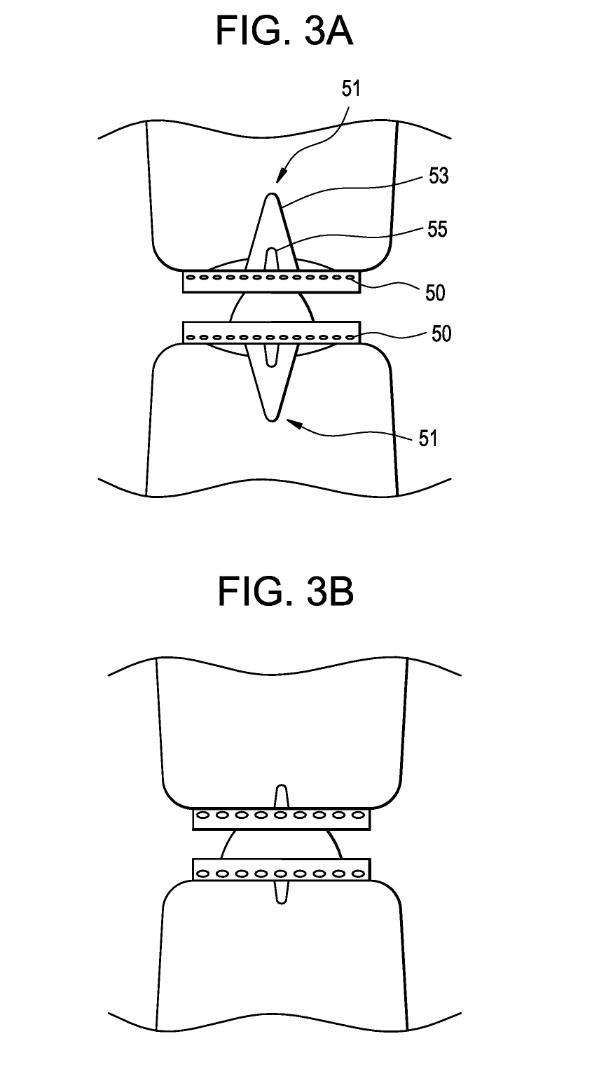

Now referring to FIGS. 3a and 3b, there is provided a depiction of a motion disc of the present invention a) immediately after its implantation in a disc space, and b) after long term follow up. The keel of this disc has an inner non-resorbable portion 55 and an outer resorbable portion 53. This disc also has a coated portion 50 on the outer surface of each endplate that is typically provided by a porous coating. FIG. 3a demonstrates how the keel 51 imparts initial stability to the implant. FIG. 3b discloses how, at long term follow up, the resorbable portion 53 of the keel has been resorbed, leaving behind only the non-resorbable portion 55. FIG. 3b also discloses how bone has grown not only into the porous surface of the endplate, but also across the chisel cut made by the resorbable portion 53 of the keel.

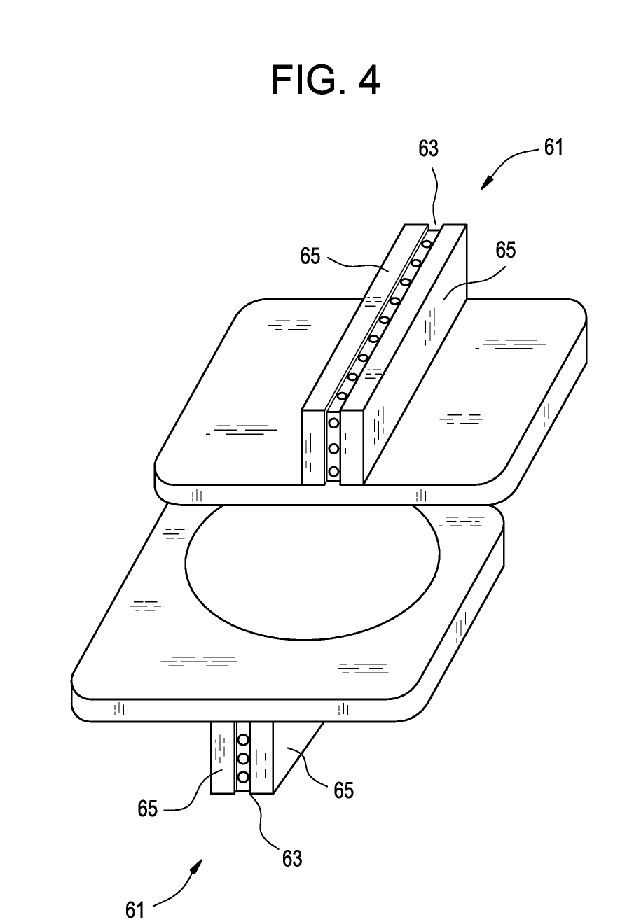

Now referring to FIG. 4, there is provided a depiction of the motion disc of the present invention having a partially resorbable keel 61 having a porous non-resorbable inner portion 63. This embodiment provides greater long term stability and fixation for the implant.

The middle porous portion 63 of this keel may be a thin porous sheet or screen or perforated layer that allows for bony ingrowth and attachment. In some embodiments, this porous middle layer can be made of titanium alloy, chrome cobalt, ceramic or a combination thereof. Similarly, the middle porous portion 63 may be a woven mesh or a chemically deposited or laser-fused network of fibers or particles. The fibers or particles may be formed from metallic or polymeric materials, including titanium alloys, cobalt chromium alloys, stainless steel alloys, PEEK, polypropylene, ceramic, or combinations thereof. In some embodiments, the porous portion of the keel has an average pore size D₅₀ of the matrix of between about 20 μm and about 500 μm, preferably between about 50 μm and about 250 μm. This level of porosity provides a suitable scaffold for bony ingrowth.

The outer portions 65 of the keel of FIG. 4 are resorbable shells that impart short-term rigidity and stability to the implant. These outer layers can be made of any resorbable polymer such as PGA, PLA, PLGA, or resorbable ceramics such as HA, and CaP. Preferably, a bone forming agent is embedded within the resorbable outer portions.

In other embodiments (not shown), the partially resorbable keel can include resorbable elements that are repeated along the anterior-posterior direction of the keel and extend in the medial-lateral direction across the keel. In some embodiments, the resorbable elements comprise a plurality of cylinders spaced along the anterior-posterior direction of the keel and extending in the medial-lateral direction across the keel to open upon the left and right side surfaces of the keel. In some embodiments, the resorbable elements comprise a plurality of slabs spaced along the anterior-posterior direction of the keel and extending both vertically in the keel and in the medial-lateral direction across the keel to open upon the left and right side surfaces and the upper surface of the keel.

The motion disc component of the present invention can be any prosthetic capable of restoring the natural motions of the intervertebral disc. In preferred embodiments, the motion disc is selected from the group consisting of an articulating disc, a cushion disc and a spring-based disc.

Preferred articulating motion devices are disclosed in U.S. Pat. Nos. 5,556,431 and 5,674,296, the specifications of which are incorporated by reference.

In some embodiments, the general structure of the articulating motion disc comprises:

a) a first prosthetic vertebral endplate comprising:
i) an outer surface adapted to mate with a first vertebral body,
ii) an inner surface having a first articulation surface,
iii) a body portion connecting the inner and outer surfaces,

b) a second prosthetic vertebral endplate comprising:
i) an outer surface adapted to mate with a second vertebral body, and
ii) an inner surface comprising a first articulation surface,

c) a core member comprising:
i) a first articulation surface adapted for articulation with the first articulation surface of the first endplate, and
ii) a second articulation surface adapted for articulation with the first articulation surface of the second endplate,
   wherein the core member is oriented to produce a first articulation interface between the first articulation surface of the first endplate and the first articulation surface of the core member, and a second articulation interface between the first articulation surface of the second endplate and the second articulation surface of the core member.

In some embodiments, the general structure of the articulating motion disc is a two piece design and comprises:

a) a first prosthetic vertebral endplate comprising:
i) an outer surface adapted to mate with a first vertebral body,
ii) an inner surface having a first articulation surface,
iii) a body portion connecting the inner and outer surfaces,

b) a second prosthetic vertebral endplate comprising:
i) an outer surface adapted to mate with a second vertebral body, and
ii) an inner surface comprising a second articulation surface,
   wherein the first and second articulation surfaces are oriented produce an articulation interface.

Preferably, the articulation interfaces form partial spheres.

The motion discs of the present invention can be adapted for use in any of the lumbar, thoracic or cervical spine regions. In some embodiments wherein the motion disc is adapted for use in the lumbar region, the three-piece design having a core is selected. In some embodiments wherein the motion disc is adapted for use in the cervical region, the two-piece design is selected.

In some embodiments, the endplate is made of a metallic material selected from the group consisting of a titanium alloy, cobalt chromium and stainless steel. Alternate embodiments may employ endplates made of polymers, such as PEEK or CFRP, or ceramics, such as zirconia-toughened-alumina.

A keel generally has a height that is at least as great as the thickness of the endplate (as measured by the distance between the inner and outer surfaces of the endplate). In some embodiments, the keel has a height that is at least twice as great as the thickness of the endplate. In some embodiments, the keel has a height that is at least three times as great as the thickness of the endplate.

In some embodiments, either the porous non-resorbable portion of the keel or the resorbable portion of the keel may be embedded with a formulation comprising a bone forming agent. The bone-forming agent may be:

a) a growth factor (such as an osteoinductive or angiogenic factor),

b) osteoconductive (such as a porous matrix of granules),

c) osteogenic (such as viable osteoprogenitor cells), or

d) plasmid DNA.

In some embodiments, the formulation comprises a liquid carrier, and the bone forming agent is soluble in the carrier.

In some embodiments, the bone forming agent is a growth factor. As used herein, the term “growth factor” encompasses any cellular product that modulates the growth or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and FGF-2) and FGF-4; members of the platelet-derived growth factor (PDGF) family, including PDGF-AB, PDGF-BB and PDGF-AA; EGFs; VEGF; members of the insulin-like growth factor (IGF) family, including IGF-I and -II; the TGF-β superfamily, including TGF-β1, 2 and 3; osteoid-inducing factor (OIF), angiogenin(s); endothelins; hepatocyte growth factor and keratinocyte growth factor; members of the bone morphogenetic proteins (BMPs) BMP-1, BMP-3, BMP-2, OP-1, BMP-2A, BMP-2B, BMP-7 and BMP-14, including rhGDF-5 (MP-52); HBGF-1 and HBGF-2; growth differentiation factors (GDFs), members of the hedgehog family of proteins, including indian, sonic and desert hedgehog; ADMP-1; bone-forming members of the interleukin (IL) family; GDF-5; and members of the colony-stimulating factor (CSF) family, including CSF-1, G-CSF, and GM-CSF; and isoforms thereof.

In some embodiments, the growth factor is selected from the group consisting of TGF-β, bFGF, and IGF-1. These growth factors are believed to promote the regeneration of bone. In some embodiments, the growth factor is TGF-β. More preferably, TGF-β is administered in an amount of between about 10 ng/ml and about 5000 ng/ml, for example, between about 50 ng/ml and about 500 ng/ml, e.g., between about 100 ng/ml and about 300 ng/ml.

In some embodiments, platelet concentrate is provided as the bone forming agent. In one embodiment, the growth factors released by the platelets are present in an amount at least two-fold (e.g., four-fold) greater than the amount found in the blood from which the platelets were taken. In some embodiments, the platelet concentrate is autologous. In some embodiments, the platelet concentrate is platelet rich plasma (PRP). PRP is advantageous because it contains growth factors that can restimulate the growth of the bone, and because its fibrin matrix provides a suitable scaffold for new tissue growth.

In some embodiments, the bone forming agent comprises an effective amount of a bone morphogenic protein (BMP). BMPs beneficially increasing bone formation by promoting the differentiation of mesenchymal stem cells (MSCs) into osteoblasts and their proliferation.

In some embodiments, between about 1 ng and about 10 mg of BMP are intraosseously administered into the target bone. In some embodiments, between about 1 microgram (μg) and about 1 mg of BMP are intraosseously administered into the target bone. Preferably, the BMP is rhGDF-5.

In some embodiments, the bone forming agent comprises an effective amount of a fibroblast growth factor (FGF). FGF is a potent mitogen and is angiogenic, and so attracts mesenchymal stem cells to the target area. It is further believed that FGF stimulates osteoblasts to differentiate into osteocytes. In some embodiments, the FGF is acidic FGF (aFGF). In some embodiments, the FGF is basic FGF (bFGF). In some embodiments, between about 1 microgram (μg) and about 10,000 μg of FGF are provided, preferably between about 10 μg and about 1,000 μg of FGF, more preferably between about 50 μg and about 600 μg of FGF. In some embodiments, FGF is administered in a concentration of between about 0.1 mg/ml and about 100 mg/ml, preferably between about 0.5 mg/ml and about 30 mg/ml, more preferably between about 1 mg/ml and about 10 mg/ml. In some embodiments, the formulation comprises a hyaluronic acid carrier and bFGF. In some embodiments, formulations described in U.S. Pat. No. 5,942,499 (“Orquest”) are selected as FGF-containing formulations.

In some embodiments, the bone forming agent comprises an effective amount of insulin-like growth factor. IGFs beneficially increase bone formation by promoting mitogenic activity and/or cell proliferation.

In some embodiments, the bone forming agent comprises an effective amount of parathyroid hormone (PTH). Without wishing to be tied to a theory, it is believed that PTH beneficially increases bone formation by mediating the proliferation of osteoblasts. In some embodiments, the PTH is a fragment or variant, such as those taught in U.S. Pat. No. 5,510,370 (Hock) and U.S. Pat. No. 6,590,081 (Zhang), and published patent application 2002/0107200 (Chang), the entire contents of which are incorporated herein in their entirety. In one embodiment, the PTH is PTH (1-34) (teriparatide), e.g., FORTEO® (Eli Lilly and Company). In some embodiments, the BFA is a parathyroid hormone derivative, such as a parathyroid hormone mutein. Examples of parathyroid muteins are discussed in U.S. Pat. No. 5,856,138 (Fukuda), the entire contents of which are incorporated herein in its entirety.

In some embodiments, the bone forming agent comprises an effective amount of a statin. Without wishing to be tied to a theory, it is believed that statins beneficially increase bone formation by enhancing the expression of BMPs.

In some embodiments, the bone forming agent is a porous matrix, and is preferably injectable. In some embodiments, the porous matrix is a mineral. In one embodiment, this mineral comprises calcium and phosphorus. In some embodiments, the mineral is selected from the group consisting of calcium phosphate, tricalcium phosphate and hydroxyapatite. In one embodiment, the average pore size of the matrix is between about 20 and about 500 μm, for example, between about 50 and about 250 82 m. In yet other embodiments of the present invention, in situ porosity is produced in the injected matrix to produce a porous scaffold in the porous portion of the keel. Once the in situ porosity is produced in the target tissue, the surgeon can inject other therapeutic compounds into the porosity, thereby treating the surrounding tissues and enhancing the remodeling process of the target tissue.

In some embodiments, the mineral is administered in a granule form. It is believed that the administration of granular minerals promotes the formation of the bone growth around the minerals such that osteointegration occurs.

In some embodiments, the mineral is administered in a settable-paste form. In this condition, the paste sets up in vivo, and thereby immediately imparts post-treatment mechanical support to the keel.

In some embodiments, the porous matrix comprises a resorbable polymeric material.

In some embodiments, the bone forming agent comprises an injectable precursor fluid that produces the in situ formation of a mineralized collagen composite. In some embodiments, the injectable precursor fluid comprises:
a) a first formulation comprising an acid-soluble type I collagen solution (preferably between about 1 mg/ml and about 7 mg/ml collagen) and
b) a second formulation comprising liposomes containing calcium and phosphate.

Combining the acid-soluble collagen solution with the calcium- and phosphate-loaded liposomes results in a liposome/collagen precursor fluid, which, when heated from room temperature to 37° C., forms a mineralized collagen gel.

In some embodiments, the liposomes are loaded with dipalmitoylphosphatidylcholine (90 mol %) and dimyristoyl phosphatidylcholine (10 mol %). These liposomes are stable at room temperature but form calcium phosphate mineral when heated above 35° C., a consequence of the release of entrapped salts at the lipid chain melting transition. One such technology is disclosed in Pederson, Biomaterials 24: 4881-4890 (2003), the specification of which is incorporated herein by reference in its entirety.

Alternatively, the in situ mineralization of collagen could be achieved by an increase in temperature achieved by other types of reactions including, but not limited to, chemical, enzymatic, magnetic, electric, photo- or nuclear. Suitable sources thereof include light, chemical reaction, enzymatically controlled reaction and an electric wire embedded in the material. To further elucidate the electric wire approach, a wire can first be embedded in the space, heated to create the calcium deposition, and then withdrawn. In some embodiments, this wire may be a shape memory such as nitinol that can form the shape. Alternatively, an electrically-conducting polymer can be selected as the temperature raising element. This polymer is heated to form the collagen, and is then subject to disintegration and resorption in situ, thereby providing space adjacent the mineralized collagen for the bone to form.

In one embodiment, the bone forming agent is a plurality of viable osteoprogenitor cells. Such viable cells, introduced into the bone, have the capability of at least partially repairing any bone loss experienced by the bone during the osteoporotic process. In some embodiments, these cells are introduced into the cancellous portion of the bone and ultimately produce new cancellous bone. In others, these cells are introduced into the cortical region and produce new cortical bone.

In some embodiments, these cells are obtained from another human individual (allograft), while in other embodiments, the cells are obtained from the same individual (autograft). In some embodiments, the cells are taken from bone tissue, while in others, the cells are taken from a non-bone tissue (and may, for example, be mesenchymal stem cells, chondrocytes or fibroblasts). In others, autograft osteocytes (such as from the knee, hip, shoulder, finger or ear) may be used.

In one embodiment, when viable cells are selected as an additional therapeutic agent or substance, the viable cells comprise mesenchymal stem cells (MSCs). MSCs provide a special advantage for administration into an uncoupled resorbing bone because it is believed that they can more readily survive the relatively harsh environment present in the uncoupled resorbing bone; that they have a desirable level of plasticity; and that they have the ability to proliferate and differentiate into the desired cells.

In some embodiments, the mesenchymal stem cells are obtained from bone marrow, such as autologous bone marrow. In others, the mesenchymal stem cells are obtained from adipose tissue, preferably autologous adipose tissue.

In some embodiments, the mesenchymal stem cells injected into the bone are provided in an unconcentrated form, e.g., from fresh bone marrow. In others, they are provided in a concentrated form. When provided in concentrated form, they can be uncultured. Uncultured, concentrated MSCs can be readily obtained by centrifugation, filtration, or immuno-absorption. When filtration is selected, the methods disclosed in U.S. Pat. No. 6,049,026 (“Muschler”), the specification of which is incorporated herein by reference in its entirety, can be used. In some embodiments, the matrix used to filter and concentrate the MSCs is also administered into the uncoupled resorbing bone. In some embodiments, bone cells (which may be from either an allogeneic or an autologous source) or mesenchymal stem cells, may be genetically modified to produce an osteoinductive bone anabolic agent which could be chosen from the list of growth factors named herein. The production of these osteopromotive agents may lead to bone growth.

In some embodiments, the osteoconductive material comprises calcium and phosphorus.

In some embodiments, the osteoconductive material comprises hydroxyapatite. In some embodiments, the osteoconductive material comprises collagen. In some embodiments, the osteoconductive material is in a particulate form.

1. An intervertebral motion disc comprising an outer surface adapted to mate with a first vertebral body, wherein the outer surface has an at least partially resorbable keel extending therefrom,
wherein the keel is partially resorbable, and comprises a non-resorbable inner portion and a resorbable outer portion.

2. The disc of claim 1 wherein the keel is a substantially fully resorbable keel.

3. The disc of claim 2 wherein the keel consists essentially of a resorbable polymer.

4. The disc of claim 3 wherein the resorbable polymer has a bone growth agent embedded therein.

5. The disc of claim 1 wherein the non-resorbable inner portion is attached to the outer surface of the disc.

6. (canceled)

7. The disc of claim 6 wherein the non-resorbable inner portion comprises a porous portion adapted for bony ingrowth.

8. The disc of claim 7 wherein the porous portion comprises a bone growth agent.

9. The disc of claim 6 wherein the resorbable outer portion consists essentially of a resorbable polymer having a bone growth agent therein.

10. The disc of claim 6 wherein each of the non-resorbable inner portion and the resorbable outer portion has a height, and the height of the non-resorbable inner portion of the keel is less than 50% of the height of the resorbable outer portion

11. The disc of claim 1 wherein the disc is an articulating disc.

12. The disc of claim 11 wherein the articulating disc comprises:
a) a first prosthetic vertebral endplate comprising:
i) the outer surface adapted to mate with a first vertebral body,
ii) an inner surface having a first articulation surface,
iii) a body portion connecting the inner and outer surfaces,
wherein the outer surface has the at least partially resorbable keel extending therefrom.

13. The disc of claim 12 wherein the inner and outer surfaces of the endplate define a thickness, and the keel generally has a height that is at least as great as the thickness of the endplate.

14. The disc of claim 11 wherein the disc is a three piece articulating disc.

15. The disc of claim 11 wherein the disc is a two piece articulating disc.

16. The disc of claim 1 wherein the disc is a cushion disc.

17. The disc of claim 1 wherein the disc is a spring disc.

18. The disc of claim 1 wherein the first prosthetic vertebral endplate is non-resorbable.

19. The disc of claim 1 wherein the first prosthetic vertebral endplate is made of a metal selected from a group consisting of titanium alloy and chrome cobalt.
